(19) United States
(12) Patent Application Publication
(10) Pub. No.: US 2023/0363890 A1
(43) Pub. Date: Nov. 16, 2023

(54) INTRAOCULAR LENS, INSERTION INSTRUMENT THEREFOR, ASSEMBLY PROVIDED THEREWITH, AND METHOD FOR MANUFACTURE

(71) Applicant: OPHTEC B.V., Groningen (NL)

(72) Inventors: Peter BIEMOLD, Groningen (NL); Harry Franciscus SIMON, Groningen (NL); Tjalke Jaddai ZIJLSTRA, Groningen (NL); Jan Hendrik DUSSELJEE, Groningen (NL); Roelof SPEELMAN, Groningen (NL)

(73) Assignee: OPHTEC B.V., Groningen (NL)

(21) Appl. No.: 18/030,802

(22) PCT Filed: Nov. 1, 2021
(86) PCT No.: PCT/NL2021/050669

§ 371 Date: Apr. 7, 2023

(30) Foreign Application Priority Data
Oct. 30, 2020 (NL) 2026797

(51) Int. Cl. A61F 2/16

(52) U.S. Cl. CPC A61F 2/1664; A61F 2002/1681; A61F 2002/169053; A61F 2240/001

(57) ABSTRACT

An intraocular lens, instrument for insertion thereof, assembly of lens and instrument, and method for manufacture of such a lens. The lens includes an optical element of a flexible optical material and provided with a substantially circular periphery; and at least one haptic element provided with a haptic form-locking element. The optical element and the at least one haptic element with the haptic form-locking element are mutually connected by connection of the haptic element to the optical material.

The invention relates to an intraocular lens to be inserted into an eye. Such a lens is usually inserted into the eye via an incision and secured there using haptic elements. Such intraocular lenses are also referred to as implant lens or artificial lens, and are implanted in a (human) eye to replace the original lens or as addition thereto.

Conventional intraocular lenses are inserted into the capsular bag through an incision, wherein the dimensions correspond to the folded dimensions of the lens. The intraocular lenses are here provided with an optical element, i.e. the optics, and haptic elements, i.e. haptics, for centering the lens in the capsule or securing it to the iris. During insertion a necessary incision is made for insertion of the lens into the eye.

EP 1 344 503 B1 describes an intraocular lens and an accompanying insertion instrument whereby the lens can be inserted into the eye. It is hereby possible to implant the lens on the front side of the iris or on the rear side of the iris. It further describes application of lenses of a flexible and deformable material to thereby make it possible to reduce the dimensions of the incision and limit the damage done to the eye. The intraocular lens is further provided with haptic elements which are connected to the optical element with an adhesive connection. The adhesive connection is here applied in order to attach the haptic elements of a first material to the optical element of a second, more flexible material. Diverse materials are hereby combined in a single intraocular lens and then inserted into the eye. This means that all individual materials must be suitable for application in the (human) eye. For this purpose strict requirements are set for these materials. A fixed connection is further obtained by gluing between the haptic elements and the optical element. This reduces the flexibility of the lens as a whole. This limits the flexibility such that folding of the lens is impeded and the incision must still have relatively large dimensions. The combination of different materials may further limit the lifespan of the lens. This results in a limitation of the utility of such a lens.

The present invention has for its object to obviate or at least reduce the above stated problems and to provide a lens usable in practice.

This object is achieved with an intraocular lens according to the invention configured to be inserted into an eye, wherein the lens comprises:
an optical element of a flexible optical material and provided with a substantially circular periphery;
at least one haptic element provided with a haptic form-locking element; and wherein the optical element and the at least one haptic element with the haptic form-locking element are mutually connected by connection of the haptic element to the optical material.

The intraocular lens according to the invention is provided with an optical element of a flexible material, such that the optical element is foldable. The optical element can hereby be inserted into the eye using an incision in the eye which is smaller than the diameter of the lens. In order to be able to secure the optical element in the eye, preferably to the iris thereof, the lens is provided with at least one haptic element. According to the invention, this at least one haptic element is provided with a haptic form-locking element wherein the optical element and the at least one haptic element are mutually connected using a form-locking element by connection of the haptic element to the optical material. By making use of a form-locking element the optical element is connected to the haptic element in effective manner without this requiring an unnecessarily rigid or fixed connection. The influence on the flexibility of the optical element is hereby minimal, and a smaller incision dimension can hereby suffice as compared to conventional iris-fixed lenses. The form-locking element forms a part of haptics and/or optics which ensures that parts are mutually connected. Connecting the haptic element to the optical material can be performed in diverse ways, for instance by inserting the haptic element into the optical material or by arranging optical material in or over the haptic element. An advantage of the intraocular lens according to the invention is that the lens does not come into contact with the chamber corner of cornea/iris, such that there is no danger of induced glaucoma in practice. This significantly reduces the risk of vision loss.

Providing the optical element from a flexible material makes it possible to fold the lens so as to enable insertion through a minimal incision. The lens is preferably constructed here such that the haptics cannot bend/deform (significantly) during folding/insertion of the lens through the incision in the eye.

In one of the currently preferred embodiments the haptic elements are cast in during the production process of the intraocular lens, optionally under pressure. A further processing is possible if desired, for instance by milling and turning the optical material in order to obtain the correct shapes and vision prescription. As an alternative to casting in a haptic element it is possible to insert the haptic element into a recess after manufacture of the optical element, wherein the haptic element fixed itself a recess arranged in the optical element. This can for instance be done using a somewhat resilient or yielding element, such that the haptic element fixes itself in the optical element.

The number of haptic elements can be adapted to the specific application and the type of intraocular lens. This number amounts to at least one, is preferably in most cases two and, if desired, can also comprise more than two haptic elements or, as further alternative, a (partially) annular haptic element. Although it is possible to apply a small amount of adhesive material, such as glue, to strengthen the connection, it is preferred that no glue is added. This has the advantage that no other type of material is applied. This makes application of the lens simpler in many situations in practice. The flexibility of the optical element is additionally preserved as much as possible by application of preferably only a form locking, i.e. form connection, between haptic element and optical element. This also prevents the risk of a glue connection becoming less strong over time, and possibly even breaking. This may for instance occur due to exposure to UV radiation. By providing the optical element with a substantially circular periphery, which is also understood to mean an oval or annular periphery, a relatively strong element is obtained, wherein no significant deformation occurs in the plane of the optical element, apart from during the folding for the purpose of insertion of the lens. In the context of the present invention the circular periphery is substantially the optical element, i.e. the so-called optical lens body. Additional elements, particularly on the sides, are possible in embodiments according to the present invention. The circular periphery reduces any tension buildup during production and/or in use, and thereby provides an effective lens.

The flexible material is preferably a hydrophobic or hydrophilic material. The optical element is for instance made of a silicone material and/or hydrophilic or hydrophobic acrylate. The haptic material is preferably slightly, or more preferably considerably, more rigid or stiff than the optical material and comprises for instance (compression moulded) PMMA or PES. Bending of the optical element is preferably performed around a central axis between two haptic elements in the plane of the optical element so as to further reduce the tension buildup. The haptic elements are preferably smaller than 3 mm and preferably amount to about 2.5 mm, wherein a sufficient retaining force of the lens to the iris is achieved and the risk of imparting (permanent) deformation during insertion is additionally counteracted.

In one of the currently preferred embodiments of the lens the optics are made of hydrophobic acrylic and the haptics of PMMA. The connection between optics and haptics is here a connection between two different materials. In such an embodiment the materials, design and connection are such that a foldable lens with sufficient strength is ensured.

As a further additional advantage, the intraocular lens according to the invention can be embodied in a refractive version, placed on the front side of the iris, to correct the vision (lens-replacing), wherein the natural lens can be preserved. This increases the utility of the lens according to the invention.

In an advantageous embodiment according to the invention the haptic element is provided with an opening or cut configured for placing the intraocular lens on both a front side and a rear side of the iris.

Providing a cut, or alternatively an opening, in the haptic element brings about an increased flexibility for a location for placing of the intraocular lens according to the invention. This is because it is possible to place the same lens on the front side (anterior chamber) and on the rear side (posterior chamber) of the iris. The lens according to the invention can hereby be utilized flexibly, and problems or errors regarding the type of lens are additionally avoided.

Owing in part to the above stated opening or cut, the haptic element is preferably suitable for placing in the eye with an attachment to the iris by means of a type of iris tissue clip mechanism which is possible with such a preferred embodiment.

In an advantageous embodiment according to the invention the haptic form-locking element of the intraocular lens is provided with an opening which lies wholly or at least partially within the periphery of the optical element and can be filled with the flexible material of the optical element.

By providing an opening in the haptic form-locking element which is filled up wholly or at least partially with a flexible material of the optical element an effective form locking or form connection is realized. In a currently preferred embodiment this is achieved by casting in the haptic element during production of the intraocular lens according to the invention. It will be apparent that it is possible to provide more than one opening per haptic element in order to further improve the form locking.

In a further advantageous embodiment according to the invention the haptic form-locking element comprises a hook-like element which is substantially wholly or at least partially surrounded by the flexible material of the optical element.

Providing a hook-like element on the haptic element realizes an effective form connection or form locking. Such a hook-like element can for instance be embodied as a T-shape. This element can be inserted by casting in, or be arranged later in a recess in the optical element using a slightly resilient or yielding part of the optical form-locking element.

The form-locking element as part of the form locking preferably lies wholly within the substantially circular periphery of the optical element. This is preferably the case for both a haptic form-locking element provided with an opening and an optical form-locking element provided with a hook-like element. By placing this form-locking element substantially wholly inside the substantially circular periphery of the optical element the dimensions of the lens can remain limited. The amount of material needed can hereby additionally also remain limited.

The optical material is preferably hydrophobic. Such hydrophobic material is for instance described in US 10,196,470 B2.

In a currently preferred embodiment the optical element comprises polymethylmethacrylate (PMMA), or in an alternative embodiment the haptic element comprises silicone material, wherein hydrophobic material is preferably applied. In a currently preferred embodiment the haptic element is provided wholly from PMMA. A flexible, and foldable, optical element is hereby obtained.

In a currently preferred embodiment according to the invention the peripheral edge of the lens is provided with an edge configuration wherein a first side is provided with a convex form and a second side is provided with a concave form. This edge can be applied in diverse types of lens, including lenses provided with convex, concave and biconvex lens forms and the like.

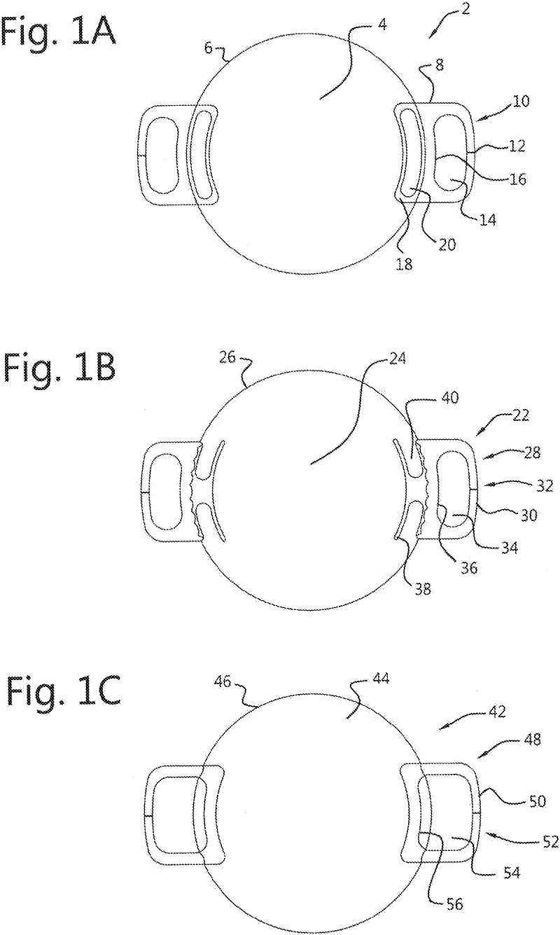

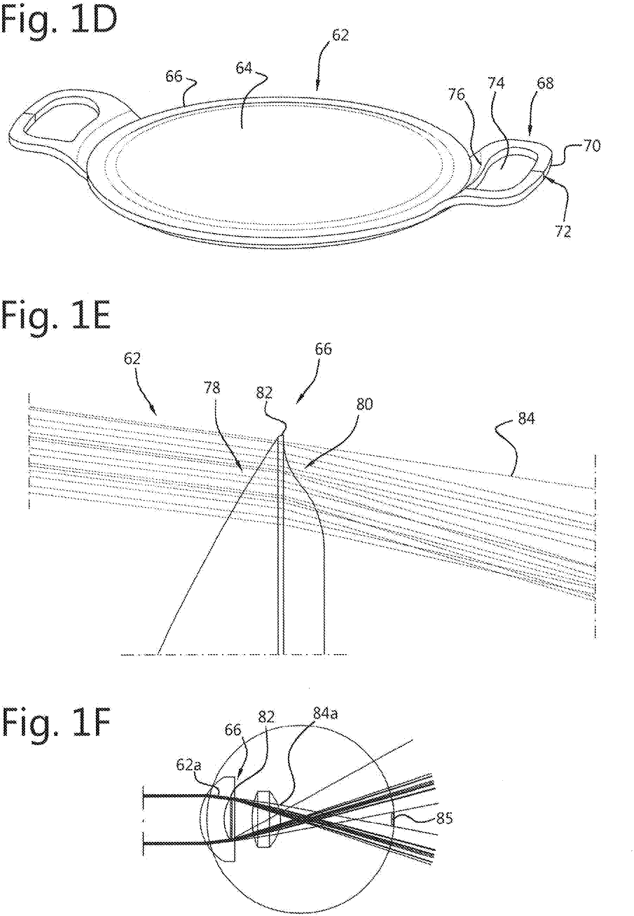

Usual edge configurations for intraocular lenses are usually straight. This can result in irritating glare and so called-halos, depending on the circumstances. Providing an edge configuration wherein use is made of a convex side and a concave side optimizes the focal point on the retina by shortening the point of incidence, whereby the irritating glare and halos are limited or are in any case prevented as far as possible. With such an edge configuration according to the invention the irritating glare and halos are in fact kept outside the focal point of the retina. This considerably improves the vision and comfort for a user provided with such an intraocular lens. An example of an embodiment according to the present invention with application of such an edge configuration is shown in FIG. 1E.

Although the edge configuration according to the invention is preferably applied in an intraocular lens according to the present invention with a form-locking element, it is also possible, as stated above, to apply such an edge configuration to other (conventional) lenses, wherein the connection between the haptic element and the optical element is for instance realized by means of an adhesive or a glue connection.

The invention also relates to an insertion instrument for inserting a lens according to the invention into an eye, wherein the instrument comprises:
a spatula configured to receive a lens;
a folding mechanism configured to fold the lens; and
a positioning mechanism configured to move the spatula for the purpose of inserting the lens.

The insertion instrument according to the invention is particularly suitable for inserting an intraocular lens into an eye. This is preferably a lens according to the invention, although it can also be a conventional lens if desired. The insertion instrument according to the invention is provided with a spatula on which or to which the lens can be attached. The spatula is used to pass the lens through the incision and to the desired location in the eye. Providing the insertion instrument with a folding mechanism enables the lens to be folded in effective manner and preferably to be held on or at the spatula in the folded state until the spatula has been passed through the incision and the folding mechanism releases the lens, such that the lens is able to unfold to the position of use. The insertion instrument is further provided with a positioning mechanism whereby the spatula can be moved and can here preferably protrude through the incision in order to position the lens in the eye.

The insertion instrument is preferably configured for single use. In a currently preferred embodiment the insertion instrument is exposed to such temperatures that it is no longer functional when it is sterilized in for instance an autoclave after use. This avoids possible problems with reuse of the insertion instrument, for instance in respect of hygiene.

The insertion instrument is further preferably provided with a clamping element at or on the spatula. The clamping element is configured to hold the lens in a folded state. The clamping element makes it possible to pass the lens through the incision in a folded state and then release it for unfolding. This results in an effective insertion instrument whereby a lens can be arranged in an eye at the desired location in an efficient manner.

In an advantageous embodiment according to the invention the insertion instrument is provided with a curved tip.

By providing a curved tip the lens can be passed through the incision to the desired location in the eye in relatively simple manner. The curved tip is preferably embodied such that the lens is also folded and the tip therefore functions as a folding mechanism. This means that the lens is introduced into the curved tip in an unfolded position of use, is here brought into a folded state during introduction into the tip and, after passing through the incision, exits the tip and can once again unfold to the position of use. This enables an effective working posture for the user and also realizes an effective, preferably passive, folding mechanism.

The tip is preferably provided with a marking for positioning the tip during insertion of the lens. The marking is preferably provided here in order to indicate the desired position of the tip relative to the incision. This increases the convenience of use for a user and avoids the risk of damage to the eye at the position of the incision as far as possible.

In an advantageous embodiment the positioning mechanism is further provided with a rotatable operating element for retracting and/or extending the spatula.

Providing a rotatable operating element provides a practical instrument for the user whereby the spatula can be extended with optimal control for the user, and can here be passed through an incision if desired. A rotating movement of the operating element is here converted into a translating movement of the spatula using a transmission. This can be performed in usual manner, preferably in the interior of the insertion instrument.

In a further advantageous embodiment according to the invention the folding mechanism of the insertion instrument comprises a folding and fixing clip.

By providing a folding and fixing clip an effective element is provided for folding of the lens. The insertion instrument with spatula, on which a lens is arranged in an unfolded state, is here preferably pressed into the clip such that the lens is forced into a folded state and the clip fixes the lens on the spatula. The clip functions here as a type of clamping element for the lens.

If desired, such a clip can be provided as a separate component of the insertion instrument and can for instance be arranged in a container, packaging or so-called blister. For the purpose of inserting the lens the clip is preferably placed close to or against the incision, after which the spatula moves relative to the clip. The front side of the spatula thereby protrudes through the incision such that the lens can unfold and can be inserted into the eye at the desired position.

In one of the currently preferred embodiments of the insertion instrument use of the clip achieves the additional advantage that the folding can be integrated with the removal of the spatula and/or instrument from the packaging. The lens is preferably pre-packaged together with the insertion instrument and provided here on the spatula. When removed from the packaging, the spatula is rotated such that the lens is pressed into the clip and is thereby folded. This is made possible by embodying the packaging such that when the insertion instrument is picked up by the handle, it will perform a pivoting/rotating movement around a rotation axis which is provided by the packaging. It is noted that, before the instrument is removed from the packaging, a user will first apply viscous material in the clip and/or to the lens in order to protect the lens. In the context of the invention viscous material is also understood to mean viscoelastic material. Owing to the preferably somewhat rotating movement of the handle of the instrument, the spatula will press the lens into a narrowed portion of the clip and the clip will preferably close around the lens and thereby fix the folded lens. The clip is for this purpose preferably provided with two mutually pivotally connected parts which are moved toward each other by the rotating movement of the instrument and fold together along with the lens. These parts are here connected at another end, for instance with a snap system, such that the clip remains closed after removal of the instrument from the packaging.

In a currently preferred embodiment a guide is provided for the purpose of the relative movement between the spatula and the clip. The guide is configured for movement thereover of the folding and fixing clip for the purpose of insertion of the lens. The guide is optionally formed by the spatula itself.

In this currently preferred embodiment the clip is slid rearward over the guide, as seen from the outermost tip of the spatula during insertion thereof. The whole relative movement between spatula and clip is here preferably brought about by the clip. During the relative movement the lens remains connected to the spatula, preferably with a clamping element, and will exit the clip as a result of the relative movement. It is hereby possible for the lens to unfold back into the position of use.

During insertion of the lens into the eye the spatula is slid into the eye, wherein the clip remains resting against the eye. Passing the front side of the spatula through the incision will cause the clip to slide rearward over the guide. The lens is hereby unfolded after entering through the incision, and can then be arranged at the desired position. The spatula can then be removed through the incision in relatively simple manner without damaging the lens and/or the tissue.

In a currently preferred embodiment the front side of the clip, i.e. the side of the clip at the outer end of the spatula, is provided with a chamfered form, wherein the chamfering particularly corresponds substantially with the contour of the eye, particularly adapted to the location where support is sought with the chamfered form. This specific shape improves the connection between the clip and the eye at the position of the incision. The front end of the spatula can be placed in the incision such that the incision will open. By placing the clip preferably against the sclera an additional support is provided at a location with good blood circulation during implantation of the lens. This has the result, among others, that the strain on the incision while the lens is being slid into the eye is minimized. This reduces the risk of damage to the eye at the position of the incision.

In an embodiment according to the present invention the positioning mechanism is provided with a pressing element for retraction and/or extension of the spatula.

Providing a pressing element enables a movement of the spatula for the purpose of insertion of the lens through the incision. This enables a good control of the movement of the spatula by the user.

The positioning mechanism is preferably further provided with a spring element. In such an embodiment, after the instrument is released, the lens is carried back into the holder of the insertion instrument after the lens has been arranged on the spatula. Use is preferably made here of a folding mechanism arranged in or on the holder for the purpose of here folding the lens, which then remains in the holder in folded state until such a time that it is desired to insert the lens into the eye.

In one of the currently preferred embodiments the spring element is provided such that the user exerting a force tensions the spring element and moves the lens into the holder. By releasing the spring element, the spring element will slacken and carry the lens out of the holder, particularly in order to move the lens to the desired position. In a correctly preferred embodiment it is possible to release the spring element in controlled manner, resulting in a slow movement of the lens, or a more complete release resulting in a more rapid movement of the lens. This increases the control of the user during insertion and reduces the risk of damage to the eye.

In a currently preferred embodiment according to the invention the insertion instrument further comprises a movable screening, such as a film, functioning as spatula.

By providing a screening, for instance in the form of a film, the lens is better protected against outside influences. The film remains present around the lens here, also in folded state of the lens. After insertion of the lens into the eye the film can be retracted from the eye in simple manner.

In a further advantageous embodiment according to the invention the folding mechanism of the insertion instrument is provided with a pinch element.

By providing a pinch element the spatula can be moved in effective manner for the purpose of inserting the lens through the incision in the eye. In one of the currently preferred embodiments the pinch element is formed by two handles which can be moved toward each other. In this embodiment the insertion instrument further comprises an operating lever for retracting and/or extending the spatula. After squeezing together the pinch element, wherein the lens is preferably folded on the spatula, the spatula can be extended using the operating lever so as to be passed through the incision into the eye. The spatula with the lens here moves out of the folding mechanism which has been activated using the pinch element. The lens can then unfold into the position of use.

In a currently preferred embodiment the operating lever is preferably provided with a rotation axis. By squeezing together the handles of the pinch element the lens is folded on the spatula. By then moving the operating lever it will rotate around a rotation axis, preferably with fixing points, such that the spatula will likewise move and is able to protrude through the incision in the eye. The rotation axis here preferably extends at least partially in a transverse direction to the spatula in order to enable the movement of the spatula in effective manner for a user.

The invention further also relates to an assembly of the insertion instrument and intraocular lens in an embodiment according to the present invention.

Such an assembly provides similar advantages and effects as described for the intraocular lens and/or the insertion instrument.

The assembly is preferably pre-packaged, after which, when it is taken into use, a quantity of viscous material is preferably added to the lens, following which the instrument is used for insertion of the lens.

In one of the currently preferred embodiments the assembly is further provided with a folding and fixing clip. In this way the folding of the lens can be realized in effective manner using the packaging of the assembly.

The spatula is preferably formed such that a lens can be pre-positioned thereon or thereat in two ways. In the case of for instance an intact natural lens, for example in the case of lens replacement, the lens is preferably pre-positioned on the spatula instead of as it were hanging down therefrom. This prevents unnecessary rotation of the lens in the eye during arranging.

The invention further also relates to a method for manufacturing a lens according to the invention. Such a manufacture can be realized in diverse ways, including so-called injection moulding and compression moulding.

The present invention further relates to a method for inserting a lens according to the invention into an eye, wherein the method comprises the steps of:
attaching the lens on or to an instrument;
placing the insertion instrument with the lens in an arranged incision; and
positioning and securing the lens.

Such a method provides similar advantages and effects as described for the lens, insertion instrument, an assembly thereof, and method for manufacturing the lens. The lens and/or the insertion instrument preferably take the form of an embodiment according to the invention. A viscous material is preferably arranged on the lens immediately before insertion in order to simplify insertion thereof and to protect the lens during insertion.

Further advantages, features and details of the invention are elucidated on the basis of preferred embodiments thereof, wherein reference is made to the accompanying drawings, in which:

FIGS. 1A-F show views of intraocular lenses according to the invention;

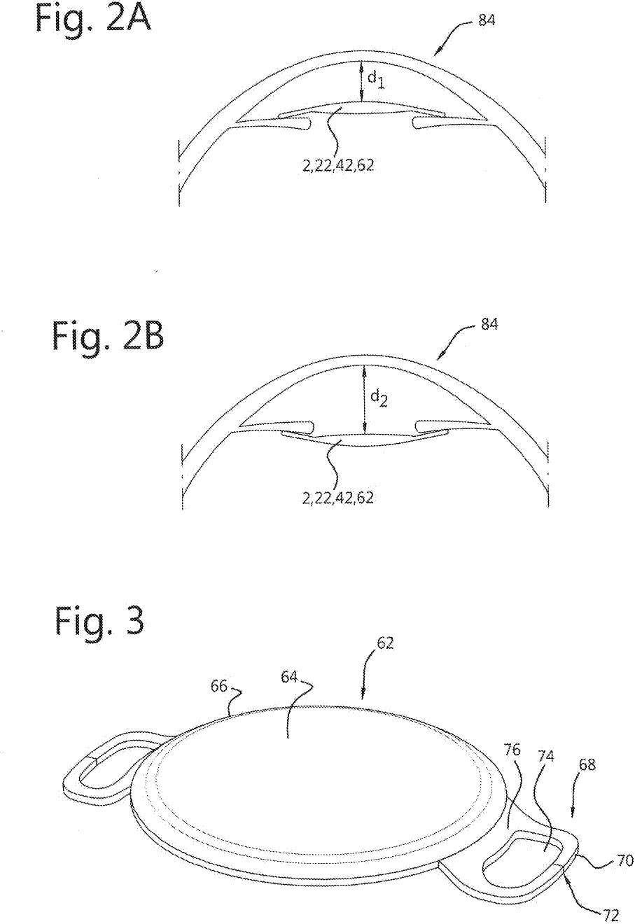

FIGS. 2A-B show views of lenses inserted into the eye;

FIG. 3 shows a view of a lens;

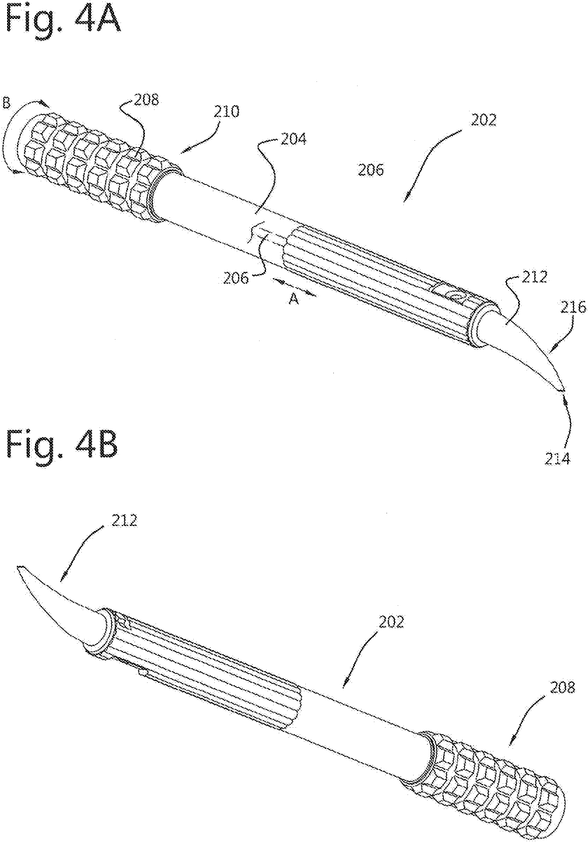

FIGS. 4A-B show a view of an insertion instrument provided with twist control;

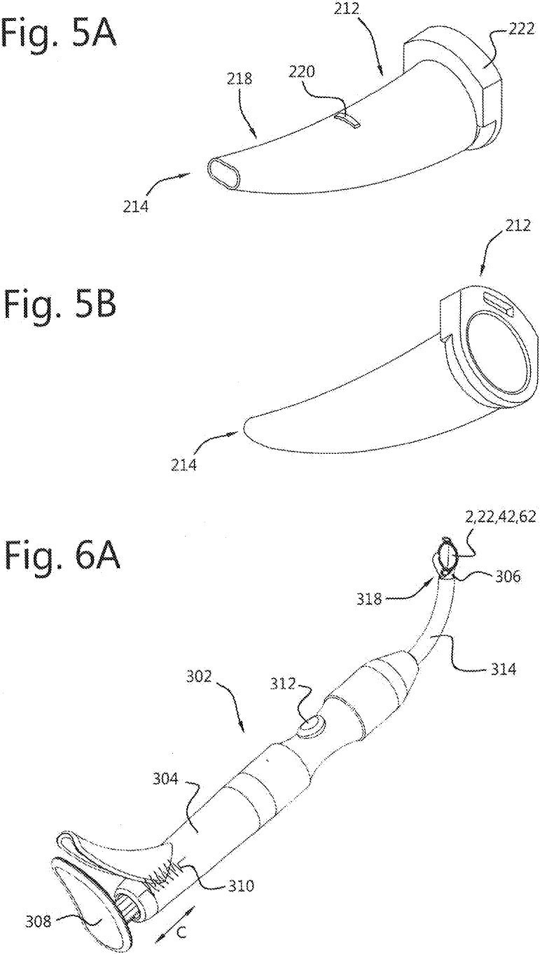

FIGS. 5A-B show views of the curved tip element of the instrument of FIGS. 4;

FIGS. 6A-B show a view of an alternative insertion instrument according to the invention provided with a press control;

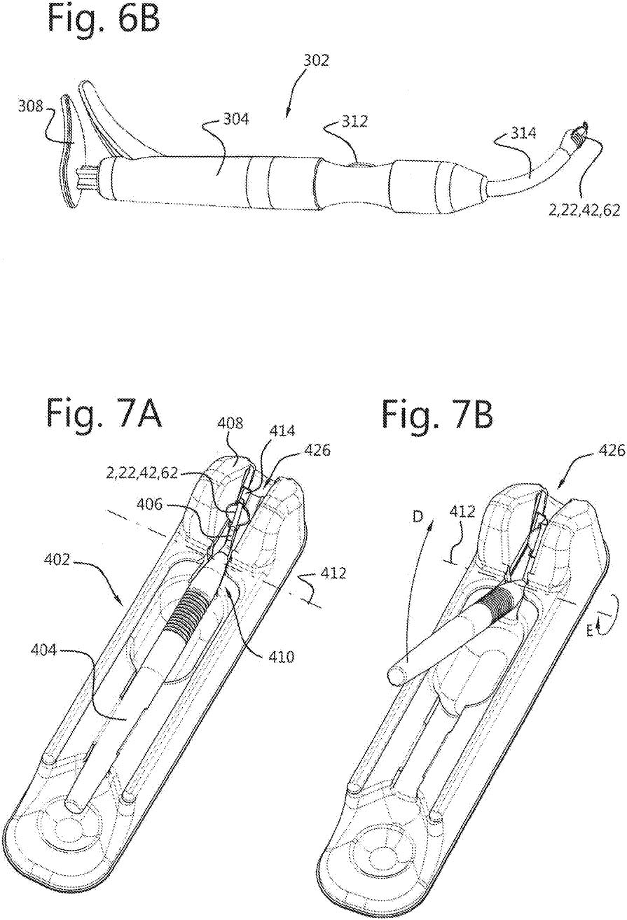

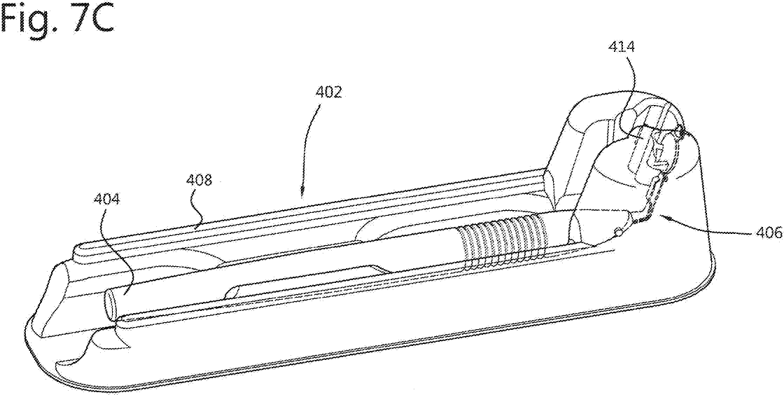

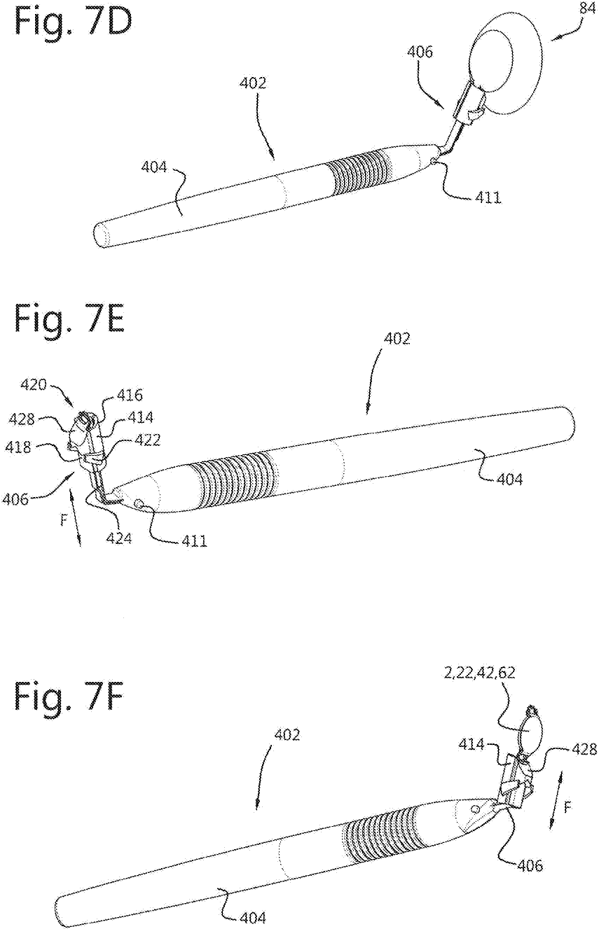

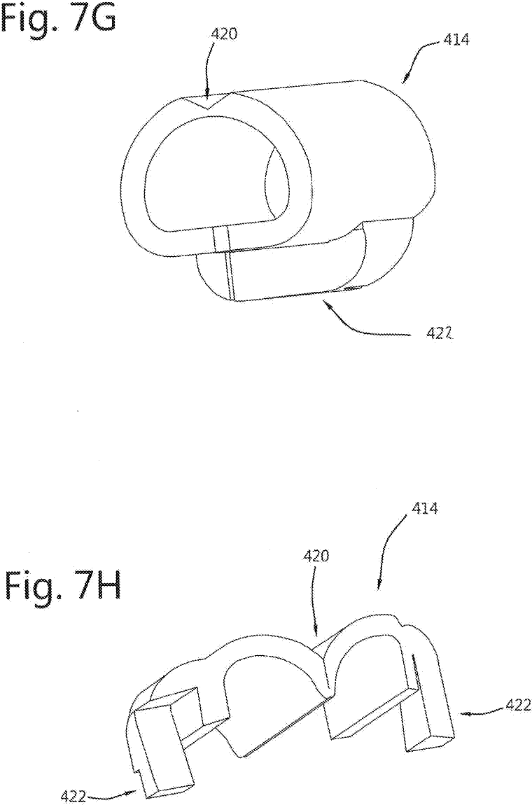

FIGS. 7A-H show a further alternative insertion instrument provided with a clip; and

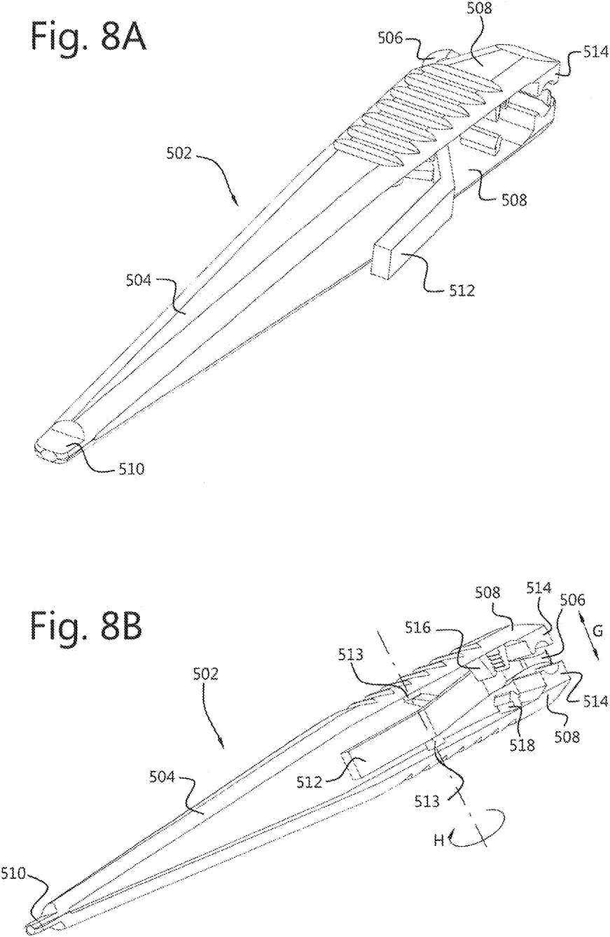

FIGS. 8A-B show a view of a further alternative insertion instrument provided with a pinch element.

Intraocular lens 2 (FIG. 1A) is provided with optics 4 with peripheral edge 6 and haptic elements 8. In the shown embodiment haptic elements 8 are provided with clamp element or hook element 10, an arranged cut 12 for imparting some flexibility to haptics 8, opening 14 and intermediate part 16. Haptic element 8 is further provided with cast-in part 18 and cast-in opening 20, wherein cast-in opening 20 is provided between cast-in part 18 and intermediate part 16. Using cast-in opening 20 in particular, an effective form-locking element is obtained. More than one opening 20 is optionally provided per haptic element 8, for instance two or three. In the shown embodiment lens 2 is preferably manufactured by casting in haptics 8.

An alternative intraocular lens 22 (FIG. 1B) is provided with optical element 24 with peripheral edge 26. Haptic element 28 is provided with clamp or hook element 30, preferably provided with cut 32, and opening 34 provided between intermediate part 36 and element 30. A number of hook-like element 38 are arranged in optical element 24, wherein space 40 is provided between hook-like element 38 and intermediate part 36.

A further alternative intraocular lens 42 (FIG. 1C) is provided with optical element 44 with peripheral edge 46. Haptic element 48 is provided with clamp or hook element 50, wherein a cut 52 is preferably provided to increase flexibility. Opening 54 is defined between hook element 50 and intermediate part 56. In this embodiment of lens 42 intermediate part 56 is cast wholly into the material of optical element 44.

In a further alternative embodiment intraocular lens 62 (FIG. 1D) is provided with optical element 64 with peripheral edge 66. In this embodiment haptic elements 68 are cast in and provided with clamp or hook element 70 provided with cut 72 to enable lens 62 to be secured. Opening 74 is provided between hook element 70 and intermediate part 76.

Peripheral edge 66 (FIGS. 1E, 1F) of lens 62 is provided on first side 78 with a convex form and provided on second side 80 with a concave form, which converge at the position of transition 82. Light rays or light beams 84 are guided such using peripheral edge 66 of lens 62 that irritating glare is kept outside the focal point of the retina and particularly macula 85 as far as possible. This achieves that only desired images are projected onto the retina. In a shown embodiment (FIG. 1F) peripheral rays 62a are guided by peripheral edge 66 with transition 82 such that rays 84a are scattered, and no undesired focal point is created.

Lens 2, 22, 42, 62 can be arranged in eye 84 in both pupillary (FIG. 2A) and retropupillary (FIG. 2B) manner. In the case of retropupillary placement d₂ is for instance about 3.33 mm, while d₁ amounts to about 2.21 mm in the case of pupillary placement. The placement of lens 2, 22, 42, 62 in eye 84 is adapted to the case of the person in question.

It will be apparent that other embodiments and combinations of shown embodiments for lens 2, 22, 42, 62 according to the invention are likewise possible.

Lens 2, 22, 42, 62 (FIG. 3) can be manufactured in diverse ways. Use can for instance be made here of a mould or mould parts. Optical material can here for instance be poured over haptic element 68. It will be apparent that alternative methods are also possible.

In a first embodiment insertion instrument 202 (FIGS. 4A-B) is provided with holder 204 in which spatula 206 is arranged for movement in direction A. Movement of spatula 206 is realized using rotating operating element 208 on which grip-enhancing element 210 is arranged in the shown embodiment. Rotation of operating element 208 in rotation direction B results in a translation movement of spatula 206 in direction A.

Arranged on the front side of tool 202 is tip 212 with outer end 214. For the purpose of inserting lens 2, 22, 42, 62 into an eye the lens will be retracted into holder 204 only once the lens has been placed on the tip of spatula 206. During retraction the lens will be folded on the spatula by narrowed portion 216 of tip 212. This preferably takes place during production so that the lens is already pre-positioned. Unfolding of the lens takes place after exiting from narrowed portion 216, which is preferably placed in or through the incision at that moment.

In the shown embodiment of tip 212 (FIGS. 5A-B), tip 212 is provided with curve 218. Curve 218 provides for an improved convenience of use for a user. In the shown embodiment marking 220 is arranged in order to indicate positioning of the lens in tip 212 for a user. Tip 212 can be connected to holder 204 of insertion instrument 202 using connecting element 222.

A lens is pre-placed on spatula 206 during use of insertion instrument 202. Alternatively, the lens has already been pre-placed on spatula 206. After a quantity of viscous material has been applied, a user will retract the lens into holder 204. Rotation of operating part 208 by the user makes it possible to place spatula 206 forward. As soon as the front side thereof protrudes from tip 212, instrument 202 can be placed in or against the incision. By then continuing rotation of operating part 208 in rotation direction B spatula 206 will protrude further through narrowed portion 214 and be moved further into the eye, such that lens 2, 22, 42, 62 will unfold and can then be positioned in the eye.

An alternative insertion instrument 302 (FIGS. 6A-B) is provided with holder 304 in which film 306 functioning as spatula is arranged. Lens 2, 22, 42, 62 can be provided at or on film 306. Insertion instrument 302 is further provided with operating element 308 which can be pressed in or otherwise moved in direction C for the purpose of moving film 306. Shown schematically is spring element 310 which is operatively connected to operating element 308 and film 306. Lens 2, 22, 42, 62 is moved inward by tensioning of spring element 310. Film 306 is here retracted into tip 314, wherein lens 2, 22, 42, 62 is folded. In the shown embodiment film 306 is provided for protection during insertion of lens 2, 22, 42, 62. A viscous material is preferably applied to the lens by the user for the purpose of retracting lens 2, 22, 42, 62 into tip 314. Movement of pressing element 308 in direction C enables spring element 310 to slacken and film 306 to extend and protrude from tip 314 through opening 318 therein, whereby lens 2, 22, 42, 62 can be inserted into the eye and can there be unfolded into the position of use.

In the shown embodiment lens 2, 22, 42, 62 is pulled inward when spring element 310 is tensioned, wherein film 306 is in this embodiment folded around lens 2, 22, 42, 62 and the lens is brought into folded state in tip 314. For the purpose of arranging lens 2, 22, 42, 62 in an eye the user will place the front side of insertion instrument 302 with tip 314 into the incision and then move pressing element 308 in direction C in order to pass spatula 306 with lens 2, 22, 42, 62 at least partially through the incision, wherein the lens will unfold and can be positioned in the eye.

A further alternative insertion instrument 402 (FIGS. 7A-H) is provided with handle 404. Spatula 406 is arranged movably. Lens 2, 22, 42, 62 can be held on the front side of spatula 406. In the shown embodiment insertion instrument 402 can be positioned in packaging or container or blister 408.

When insertion instrument 402 is removed from packaging 408, the user will move holder 404 upward in direction D. In the shown embodiment instrument 402 is here arranged with fixing points 411 in somewhat clamping manner between clamping elements 410 of packaging 408, such that a rotating movement around rotation axis 412 defined by elements 410 is performed in rotation direction E. Lens 2, 22, 42, 62 can be arranged on or at spatula 406, or at least the tip thereof.

Owing to the movement in directions D, E, insertion instrument 402 is pressed downward into clip 414 which is provided in packaging 408 in the shown embodiment. Clip 414 is provided with first part 416 and second part 418. Parts 416, 418 are pivotally connected on one side via hinge element 420 and provided on the side with snap element 422 in order to thereby be fixed and be able to hold lens 2, 22, 42, 62 in a folded state. In the shown embodiment spatula 406 is provided with guide 424, wherein in the shown embodiment spatula 406 functions as guide, whereby clip 414 can be slid in direction F relative to spatula 406.

For the purpose of arranging lens 2, 22, 42, 62 using insertion instrument 402 a quantity of viscous material is preferably first applied to the lens by a user. Holder 404 is then picked up and rotated in direction D, wherein insertion instrument 402 will rotate around rotation axis 412 in rotation direction E. Spatula 406 with lens 2, 22, 42, 62 is hereby pressed into clip 414 (FIG. 7G for closed position and FIG. 7H for open position) which will fold up using hinging element 420 and will snap together using snap connection 422, such that lens 2, 22, 42, 62 is inserted into clip 414 in a folded state. By integrating the folding process with removal of hand instrument 402 from packaging 408 instrument 402 can take a relatively simple form, whereby a better view and more control are also possible for the user during insertion and implantation of the lens. In the shown embodiment this folding up of the clip is realized by arranging a narrowed portion 426 in packaging 408. The instrument which has been removed from packaging 408 is then brought to the incision in eye 84. Base 428 is here preferably placed against the eye for additional support during implantation. Base 428 is for this purpose provided with a shape corresponding substantially with the shape of eye 84 in order to optimize the support and minimize strain on the incision. By then pressing spatula 406 through the incision clip 414 will as it were slide back in direction F via guide 424 over spatula 406. The tip of spatula 406 with lens 2, 22, 42, 62 will protrude through the incision in the eye, where the lens will unfold. Spatula 406 can then be safely removed from the incision.

Lens 2, 22, 42, 62 can optionally be attached to spatula 406 in two ways. In the case of so-called lens replacement, wherein the natural lens of the eye is intact, lens 2, 22, 42, 62 is preferably pre-positioned on spatula 406 instead of as it were hanging down therefrom. This prevents undesired rotation of lens 2, 22, 42, 62 in the eye.

In a further alternative embodiment insertion instrument 502 (FIGS. 8A-B) is provided with holder 504 in which spatula 506 is arranged. Pinch elements 508 are movable in direction G owing to flexible connection 510. Folding elements 514 are moved toward or away from each other in direction G using pinch elements 508. Snap elements 516, 518 are provided to fix pinch elements 508 relative to each other.

When inserting lens 2, 22, 42, 62 arranged on the front side of spatula 506, instrument 502 is removed from the packaging and viscous material is preferably applied to the lens by a user. By squeezing together pinch elements 508 the lens is folded on spatula 506 with folding elements 514 and preferably fixed with snap elements 516, 518. Instrument 502 is then placed in front of the incision in the eye. By pressing operating lever 512 a rotation movement will be performed around pivot points 513 in direction H, whereby spatula 506 moves out of folding elements 514 and protrudes through the incision in the eye, whereby the lens will unfold and can be position. The user can then retract the instrument.

The present invention is by no means limited to the above described preferred embodiments thereof. The rights sought are defined by the following claims, within the scope of which many modifications can be envisaged. The shown embodiments of the insertion instrument can thus for instance also be applied to more conventional lenses.

1-25. (canceled)

26. An intraocular lens configured to be inserted into an eye, the lens comprising:
an optical element of a flexible optical material and provided with a substantially circular periphery; and
at least one haptic element provided with a haptic form-locking element; and
wherein the optical element and the at least one haptic element with the haptic form-locking element are mutually connected by connection of the haptic element to the optical material.

27. The intraocular lens of claim 26, wherein the haptic element is provided with an opening or cut configured for placing the intraocular lens on both a front side and a rear side of the iris.

28. The intraocular lens of claim 26, wherein the haptic form-locking element comprises an opening which lies wholly or partially within the periphery of the optical element and can be filled with the flexible material of the optical element.

29. The intraocular lens of claim 26, wherein the haptic form-locking element comprises a hook-like element which is wholly or partially surrounded by the flexible material of the optical element.

30. The intraocular lens of claim 28, wherein the form-locking element lies substantially wholly within the substantially circular periphery of the optical element.

31. The intraocular lens of claim 30, wherein the optical material is hydrophobic, and wherein the haptic material comprises compression moulded polymethylmethacrylate (PMMA).

32. The intraocular lens of claim 26, wherein the peripheral edge of the lens is provided with an edge configuration wherein a first side is provided with a convex form and a second side is provided with a concave form.

33. An insertion instrument for inserting the lens according to claim 26 into an eye, the instrument comprising:
a spatula configured to receive a lens;
a folding mechanism configured to fold the lens; and
a positioning mechanism configured to move the spatula for the purpose of inserting the lens.

34. The insertion instrument of claim 33, wherein the spatula is provided with a clamping element configured to hold the lens in a folded state.

35. The insertion instrument of claim 33, provided with a curved tip.

36. The insertion instrument of claim 35, wherein the tip is provided with a marking for positioning the tip during insertion of the lens.

37. The insertion instrument of claim 34, wherein the positioning mechanism is provided with a rotatable operating element for retracting and/or extending the spatula.

38. The insertion instrument of claim 34, wherein the folding mechanism comprises a folding and fixing clip.

39. The insertion instrument of claim 38, wherein the positioning mechanism comprises a guide which is configured for movement thereover of the folding and fixing clip during insertion of the lens.

40. The insertion instrument of claim 34, wherein the positioning mechanism is provided with a pressing element for retraction and/or extension of the spatula, wherein the positioning mechanism further comprises a spring element.

41. The insertion instrument of claim 34, further comprising a screening functioning as spatula and/or wherein the folding mechanism is provided with a pinch element and further comprising an operating lever for retracting and/or extending the spatula, wherein the operating lever is provided with fixing points.

42. The insertion instrument of claim 36, wherein the insertion element is provided with an intraocular lens to provide an assembly of insertion instrument and intraocular lens.

43. The insertion element of claim 42, further comprising a folding and fixing clip.

44. A method for manufacturing the lens of claim 26.

45. The method of claim 44, for inserting a lens according to any one of the foregoing claims in an eye, the method comprising the steps of:
attaching the lens on or to an instrument;
placing the insertion instrument with the lens in an arranged incision; and
positioning and securing the lens.
